(19) United States
(12) Patent Application Publication
(10) Pub. No.: US 2007/0042372 A1
(43) Pub. Date: Feb. 22, 2007

(54) Method for designing dna codes used as information carrier

(75) Inventor: Masanori Arita, Tokyo (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: NATIONAL INSTITUTE OF ADVANCED INDUSTRIAL SCIENCE AND TECHNOLGY, TOKYO (JP)

(21) Appl. No.: 10/558,502

(22) PCT Filed: May 27, 2004
(86) PCT No.: PCT/JP04/07271

§ 371 Date: Aug. 16, 2006

(30) Foreign Application Priority Data
May 29, 2003 (JP) 2003-151738

(51) Int. Cl. C12Q 1/68; G06F 19/00; C07H 21/04

(52) U.S. Cl. 435/6; 536/23.1; 702/20

(57) ABSTRACT

The present invention provides a method for designing DNA code consisting of a set of information codes as an information carrier to write optional information into an optional noncoding region not including any DNA genetic information which can avoid an error occurring when the designed DNA is used. A set S1 of the base sequences corresponding to a signal unit for information transmission is obtained as follows: 1) selecting a template such that its Hamming distance of templates, against its block shift, and against the ligated sequences are equal to or above the predetermined value, when DNA sequence of predetermined length is specified by the binary string of 0 and 1 (template), meaning that the position of G or C ([GC]), or A or T ([AT]) are fixed, 2) further selecting a template having a subword constraint of length m from the set of the selected templates, and 3) combining thus selected template and codewords of the predetermined error-correcting codes having a subword constraint of length m.

### TECHNICAL FIELD

The present invention relates to a method for designing a DNA code which can be a simple, general information carrier for writing information into biopolymers as well as which can avoid errors occurring when artificially designed DNA is used as an information carrier, a DNA code obtained by the method for designing, and a technique for writing optional information into DNA by embedding the DNA codewords into an optional noncoding region not including any genetic information.

### BACKGROUND ART

DNAs have a structure wherein four types of base, that is, adenine (A), cytosine (C), guanine (G) and thymine (T), are ligated together like a strand. Since A and T, and C and G form base pairs by hydrogen bond respectively, A-T and C-G are considered to be complementary. The two DNA strands have a complementary double helix structure, and the DNA double helix is separated into single-stranded DNAs when temperature rises, and the single-stranded DNAs bind to complementary strands again when temperature drops. This process of binding to complementary strands is called hybridization, and it is well known that the temperature at which DNA strands separate or hybridize depends on GC content in the sequence. Further, a noncomplementary base pair in a double strand cannot form stable hydrogen bond and it is called a (base) mismatch. The stability (e.g. free energy) of a DNA double helix depends on the number and distribution of base mismatches (see e.g. Biochemistry 37, 26, 9435-9444, 1998). Plural oligonucleotide sequences corresponding to the letters are prepared in order to write information by using this DNA. A set of artificial oligonucleotide sequences of fixed length is used in many fields of application as set forth below.

For instance, as biotechnology advances, artificial gene engineering is performed routinely; protecting the copyright of the modified gene has been emphasized. However, a gene has no major feature particularly except that it is constituted by combination of 4 bases, and the method for characterizing the cells of organisms, gene fragments, or the like which are newly generated by gene engineering to protect them from abuse, has not been established yet. In order to limit the use or piracy unintended by the developers, DNA signature or DNA steganography (an externally invisible signature, achieved by hiding the signature in the other information) is regarded as useful. It is actualized by, for instance, denoting the information with signature as a DNA base sequence to locate the origin of the DNA, and incorporating the base sequence for location into artificially modified genome (see, e.g. Japanese Laid-Open Patent Application No.2001-352980). Oligonucleotide sequences of fixed length are artificially designed and used as sequences for signature in practical use.

In addition, there is quite a new computation called “DNA computation”, representing computing paradigms unlike the current computation (see e.g. Science 266, 5187, 1021-1024, 1994) In this field of study, symbol processing is realized by denoting logical variables or graph components as base sequences of DNA for solving mathematical problems and applying experimental methods in molecular biology to the base sequences. A set of artificially designed oligonucleotide sequences of fixed length is used here, too.

Moreover, DNA tag/antitag system (see, e.g. Proceedings of the National Academy of Sciences of USA 89, 12, 5381-5383, 1992, Proceedings of the National Academy of Sciences of USA 97, 4, 1665-1670, 2000, and Journal of Computational Biology 7, 3-4, 503-519, 2000), is used for monitoring gene expressions with the use of oligonucleotide tags of fixed-short length. These tags can be regarded as codes denoting information corresponding to respective genes. Other than this system, a method for using DNA as a future medium for data storage (see, e.g. 10^{th} Foresight Conference on Molecular Nanotechnology (Bethesda, USA) Poster abstract, 2002) has been also advocated. Oligonucleotide sequences of fixed length are used for denoting respective data in these approaches, too.

All of the above techniques intend to write information into base sequence and require design of “DNA codes”. Here, the DNA code is a set of base sequences different from each other but having the same length. The constraints that thus designed DNA codes should satisfy are following: all codewords (base sequences) must have constant physical properties such as melting temperature, and they do not induce unwanted hybridization (mishybridization) between codewords, and the method for designing has much in common with the method for designing the classical error-correcting codes. However, design of DNA code is different from that of error-correcting codes in some points; there is no standard method for designing codewords. Three basic approaches which have been used for design of DNA codewords conventionally are described below: (1) the template-map strategy, (2) De Bruijn construction, and (3) the stochastic method.

(Template-Map Strategy)

This method for designing was first proposed by Condon's group (see, e.g. Nucleic Acids Research 25, 23, 4748-4757, 1997). The basic idea is to divide constraints on the DNA code and separately assign them into two binary codes, and to combine them together to constitute a quaternary code (a DNA code). For instance, one binary code (called a template) keeping GC content constantly and the other binary code (called a map) ensuring mismatches between any codewords, are combined to design a quaternary code which fulfills both constraints. Frutos et al. designed 108 words of DNA codes of length 8 to have following features: (1) each codeword has four GCs, and (2) there are at least four mismatches between each of codewords, including complementary sequence (see, e.g. Nucleic Acids Research 25, 23, 4748-4757, 1997). Further, Li et al., used the Hadamard code, generalized this method for designing to longer DNA code (see, e.g. Langmuir 18, 3, 805-812, 2002). They presented, as an example, the design of 528 words of DNA code of length 12 with six minimum mismatches.

As a DNA code is produced by combining two binary codes in the template-map strategy, the DNA code designed by using this technique can only fulfill the properties which are studied with binary codes, conventionally. However, DNA, unlike the code used electronically, cannot specify the comma of codewords, therefore, it is necessary to have the system to necessarily detect the shift when a reading frame of codeword is shifted. The property is referred to as comma-free since it does not need comma. A code necessarily producing d number of mismatches (when the reading frame is shifted) between concatenation of a codeword and each codeword is referred to as a comma-free code of index d. Unfortunately, a theory regarding comma-free codes of high index has seldom been studied in binary codes. Therefore (see, e.g. IEEE Transactions on Information Theory, IT-11, 107-112, 1965, and Stiffler, J. J., Theory of Synchronous Communication. Prentice-Hall, Inc., Englewood Cliffs, N.J., 1971), comma-freeness cannot be conferred to DNA codes in the template-map strategy.

(De Bruijn Construction)

The longer a consecutive run of matched base pairs, the higher is the risk of mishybridization. Accordingly, it is necessary to impose a constraint (a subword constraint) without a consecutive bases match of length k (k: generally 7 to 8). Ben-Dor et al. showed an optimal choosing algorithm of oligonucleotide tags that satisfy the subword constraint of length k by cleaving a sequence of length k sharing the same melting temperatures from De Bruijn sequence of order k (see, e.g. Journal of Computational Biology 7, 3-4, 503-519, 2000). De Bruijn sequence of order k is a circular sequence of length 2^{k} in which each of sequences of length k occurs exactly once. A linear time algorithm for the construction of a De Bruijn sequence is known.

There are other similar techniques using a De Bruijn sequence and DNA chips using the tags constructed in this manner are commercially available (see, e.g. European Patent No.97302313 and Genome Research 10, 6, 853-860, 2000).

The oligonucleotide sequence chosen from the De Bruijn sequence of order k does not have a consecutive match of length k or longer, therefore, a DNA codeword of length 2k or longer can avoid a complete match of the concatenation of a codeword with the other codeword (a comma-free code of index 1). In fact, Brenner applied the comma-free code of index 1 to the design of oligonucleotide tags (see, e.g. U.S. Pat. No. 5,604,097, Proceedings of the National Academy of Sciences of USA 89, 12, 5381-5383, 1992, and Proceedings of the National Academy of Sciences of USA 97, 4, 1665-1670, 2000). However, it is difficult to confer comma-free codes of index 2 or more, when the De Bruijn sequence is used. Further, it is also difficult to guarantee the number of mismatches between codewords designed with the use of De Bruijn sequence. Therefore, it is highly difficult to design DNA codes having high comma-freeness of index and large number of mismatches between codewords.

(Stochastic Method)

The stochastic method is the most widely used approach in code design. Deaton et al. used genetic algorithms to find codewords sharing similar melting temperatures as well as satisfying the ‘extended’ Hamming constraint, i.e. a constraint where mismatches in the case of shift are also considered (see, e.g. DNA Based Computers II, DIMACS Series in Discrete Mathematics and Theoretical Computer Science 44, 247-258, 1998). According to their report, due to the complexity of the problem, genetic algorithms can only be applied to design of the codewords of up to length 25 (see, e.g. Proceedings of the 3^{rd} Annual Genetic Programming Conference, Morgan Kaufmann 684-690, 1998).

Landweber et al. used a random codeword-generation program to design two sets of 10 codewords of length 15. Thus designed sequence satisfies following conditions: (1) no more than five consecutive base matches in ligation of any codewords, (2) standardized melting temperatures of 45° C., (3) avoidance of secondary structures, and (4) no consecutive combinations of more than seven base pairs (the fourth condition is not necessary when the first condition is satisfied. Here, conditions appearing in the original text are shown.). They realized these constraints with only three types of bases (see, e.g. Proceedings of the National Academy of Sciences of USA 97, 4, 1385-1389, 2000). Other groups who designed codewords with only three types of bases likewise employed random codeword-generation for design (see, e.g. DNA Computing: 6^{th} International Workshop on DNA-Based Computers (DNA 2000; Leiden, The Netherlands), LNCS 2054, 17-26, 2001, and Science 296, 5567, 499-502, 2002).

Although no theoretical analysis for algorithms used in stochastic method has been performed yet, the power of the technique is evident in the work of Tulpan et al. (see, e.g. Proceedings of 8^{th} International Meeting on DNA-Based Computers (DNA 2002; Sapporo, Japan), 311-323, 2002). By using the stochastic method, they could increase the number of codewords designed by the template-map strategy, while they failed in outperforming the design by the template-map strategy with the use of the stochastic method alone. Therefore, it is preferable to apply the stochastic method for increasing the number of already designed codewords. Defects of the stochastic method are exemplified as follows: the designed codeword differs every time it is designed (since it is stochastic), the number of codewords which can be designed cannot be assumed, and the feature (e.g. the number of mismatches) of the codeword to be designed cannot be assumed in advance.

Conventional methods for designing are shown as set forth above, all of which have defects, so they cannot be the ideal methods for designing. The ideal codewords should satisfy the various constraints described below.

(Hamming Distance Constraints)

Designed DNA codes should keep a large Hamming distance between all codewords. What makes the DNA code-design more complicated comparing to the theory of error-correcting code is that the number of mismatches in the hybridization not only with the codewords but also with their complementary sequences must be considered.

(Comma-Free Constraints)

Comma-freeness is referred to as a property which guarantees the predetermined number of mismatches not only when the reading frames of the codewords are overlapped but also when the reading frames of the sequence are shifted. Since DNA does not have a fixed reading frame, it is desirable that the designed code is comma-free. By definition, a code is comma-free of index d when the concatenation of codewords x₁ x₂ . . . xₙ and y₁ y₂ . . . yₙ, (i.e. xᵣ₊₁ xᵣ₊₂ . . . xₙ y₁ y₂ . . . yᵣ; 0<r<n), which are any 2 codewords not necessarily different, has necessarily d or more of mismatches with the other codeword (see, e.g. Canadian Journal of Mathematics 10, 202-209, 1958, and Canadian Journal of Mathematics 39, 3, 513-526, 1987). Thus, DNA codewords should be comma-free of high index. Here, it should be noted that the property of comma-freeness is not compensated by introducing ‘spacer’ codewords between codewords. Presence of the spacers may facilitate decoding codewords, but it does not contribute to the avoidance of mishybridization. Moreover, spacers lower its information content as they introduce excess DNA sequences between each codeword.

(Energy Constraints)

In addition to the above constraints on mismatches, the melting temperatures of DNA codes are necessarily to be standardized for guaranteeing the unbiased behavior in experiments. There are several formulas to estimate the melting temperature: (1) for very short oligonucleotides, the GC content or the 2-4 rule (in the 2-4 rule, melting temperature is estimated as (the number of AT base pairs)×2+(the number of GC base pairs)×4° C.), (2) for relatively short oligonucleotides, the approximation using the nearest neighbor base pair method (see, e.g. Proceedings of the National Academy of Sciences of USA 83, 11, 3746-3750, 1986 and Biochemistry 37, 26, 9435-9444, 1998), and (3) for longer oligonucleotides, Wetmur's approximation (see, e.g. Critical Reviews in Biochemistry and Molecular Biology 26, 3-4, 227-259, 1991). Using one of these formulas, all codewords can be designed so that their melting temperatures are within a narrow range.

(Other Constraints)

Following constraints in terms of base mismatches, depending on the model used, are known.
1. Subsequences corresponding to restriction sites, simple repeats of bases, or other biological signal sequences, should not appear. This constraint should not appear anywhere in concatenation of them (including their complementary sequence) as well as in designed codewords. This constraint will be necessary when the codeword is written into the predetermined sequence such as genomic DNA, or when the specific restriction enzyme is used.
2. Any subword of length k should not appear more than once between the designed codewords and their concatenation. This constraint is necessary to ensure the avoidance of mishybridization.
3. A secondary structure that impedes expected hybridization of codewords should not arise. This constraint is necessary when temperature control plays an important role in application field of DNA codewords.

### DISCLOSURE OF THE INVENTION

### Problems to be Solved by the Invention

As aforementioned, as bio- and nano-technology advances, the demand for writing information into DNA increases. The field to which the technique is applied is unlike conventional biotechnology in that artificial information is tried to be written into DNA. Although various design strategies for DNA code have been proposed, the aim of those strategies is not providing the standard code (like the ASCII code) for using DNA as an information carrier. Presumably, it is because constraints to be satisfied by DNA sequences depend on the fields where the respective strategies are used. A simple, versatile code is required when DNA is used as an information carrier.

When information is written or read in DNA, following phenomena should be taken into account.
1. Errors such as misreading of base sequence or skip of some bases occur when DNA is sequenced.
2. A specific sequence referred to as a primer is necessary for sequencing DNA. Primer sequences, aligned at the both ends of the sequence preserving information, amplify only the region (an information sequence) between the primer sequences.
3. The physical properties (e.g. melting temperatures) of the sequences to be written into DNA should be standardized. When the physical properties are widely different depending on the DNA sequences to denote information, a specific secondary structure is formed or amplification efficiency by the primers is sharply reduced. Further, the information sequence is incorporated into the object DNA with difficult, too.
4. There is a sequence whose appearance is not preferable. Therefore, a constraint which prevents the specific restriction site from appearing in the information sequences, and a constraint which prevents having the common sequence with the specific genetic sequence, are very important and common.

The technique regarding conventional DNA code does not consider misreading, since the theory thereof is constructed based on the hypothesis that written information can be sequenced from DNA “in its entirety”. Further, it does not consider primers as well or it merely proposes a very ambiguous solution such as “preparing specific sequences at the both ends of the information to be embedded into DNA”. In addition, the conventional method does not show specific means for writing information into DNA, accordingly, it does not indicate techniques for standardizing the physical properties and preventing the appearance of the specific sequence, too. There are a number of experimental constraints for replication of genetic information, so even high level of technology does not enable replication of genetic information without any errors. Further, even if errors can be eliminated at replication stage, mutation of the sequence by biomolecule or radiation should be considered when the information sequence is written into DNA of living body.

Therefore, the object of the present invention lies in provision of a method of designing a set of base sequences for codes (a set of symbols which are given meanings artificially by alphabet or the like), used as information carriers to read or write optional information into optional noncoding regions not including any DNA genetic information, i.e., a method of designing DNA codes. The codewords of the DNA codes can correspond to the code- system used by computer, and they have characteristics in that any arrangement of the letters permits decode of codewords with very high reliability. This DNA codeword, having features utterly different from those of natural DNA, can be embedded into an optional area not including any DNA genetic information. Further, the DNA codewords prepared by the method for designing of the present invention can also be utilized as a storage media of information.

### Means for Solving the Problems

The inventor previously proposed: a method for systematically designing a set S1 of oligonucleotide sequences of predetermined length n (n is an integer, 3 or more, preferably, 6 or more), wherein each of oligonucleotide sequences in the set S1 induces equal to or more than a fixed number of mismatches against any of oligonucleotide sequences in the set S1, complementary sequences of each of oligonucleotide sequences in the set S1, sequences constructed by shifting these sequences, and sequences produced by ligation of these oligonucleotide sequences, of their complementary sequences, and of the oligonucleotide sequences and their complementary sequences, wherein the set S1 of oligonucleotide sequences can avoid mishybridization between any of said oligonucleotide sequences, said complementary sequences, sequences constructed by shifting these sequences, and sequences produced by ligation of said oligonucleotide sequences, of said complementary sequences, and of said oligonucleotide sequences and said complementary sequences; and a method for systematically designing a set S1 of oligonucleotide sequences which can avoid mishybridization for reverse sequences as well as for complementary sequences (Japanese Patent Application No. 2001-331732).

The present inventor has conducted an intensive study to solve the above-identified problem, as it is necessary not only to maintain error-correcting function but also physical property such as meting temperatures homogenous for design of sequences to embed information into DNA, the inventor found a method for designing DNA code satisfying all these conditions by following steps: further selecting a template having a subword constraint of length m from the templates used in designing the above-mentioned set of oligonucleotide sequences by the present inventor, and combining it with codewords of predetermined error-correcting codes having also a subword constraint of length m to make them a set of S2 of base sequences which can be used as letters in describing information, and the present inventor realized the correspondence of a conventional code system including ASCII and a code system by DNA base sequence. The present invention has thus been completed.

That is, the present invention provides: a method for designing a DNA code, comprising the following steps: 1) selecting a binary string (GC templates) such that all of its Hamming distance against its reverse sequence, its block shift, and the distance against the overlap part of its tandem concatenation, its concatenation with its reverse sequence, and the tandem concatenation of its reverse sequence are equal to or above the predetermined value k, and in the following, an oligonucleotide sequence of predetermined length n (n is an integer 6 or more) is specified by the binary string of 0 and 1 (GC template) of predetermined length L (L is an integer 6 or more), meaning that the position of G or C ([GC]), or A or T ([AT]) are fixed; 2) selecting a set having a subword constraint of length m as a template from the set of the selected GC templates; and 3) constructing a set S1 of the oligonucleotide sequences by combining codewords of the predetermined error-correcting codes having a subword constraint of length m likewise (“1”); a method for designing a DNA code, comprising following steps: 1) selecting a binary string (AG template) such that its Hamming distance against its reverse inverted sequence, its block shift, and the distance against the overlap part of its tandem concatenation, its concatenation with its reverse inverted sequence, and the tandem concatenation of its reverse inverted sequence are equal to or above the predetermined value k, and in the following, an oligonucleotide sequence of predetermined length n (n is an integer 6 or more) is specified by the binary string of 0 and 1 (AG template) of predetermined length L (L is an integer 6 or more), meaning that the position of A or G ([AG]), or T or C ([CT]) are fixed; 2) selecting a set having a subword constraint of length m as a template from the set of the selected AG templates; and 3) constructing a set S1 of oligonucleotide sequences by combining the codewords of predetermined error-correcting codes having a subword constraint of length m likewise (“2”); a method for designing a DNA code, wherein any of oligonucleotide sequences of the set S1, of which Hamming distance is kept equal to or above k, induces mismatches equal to or above the predetermined value against any of the sequences, their complementary sequences, sequences constructed by shifting these sequences, and sequences produced by ligation of sequences in the set S1, of their complementary sequences, and of the sequences and their complementary sequences, and wherein the sequence in the set S1 can avoid mishybridization between them, their complementary sequences, sequences constructed by shifting these sequences, and sequences produced by ligation of the sequences in the set S1, of their complementary sequences, and of the sequences and their complementary sequences, which facilitates decoding information (“3”); a method for designing a DNA code, wherein the set S1 of oligonulcleotide sequences of predetermined length n is a set S1 of oligonucleotide sequences of length 32 or less (“4”); a method for designing a DNA code, wherein the predetermined value k of Hamming distance is one-fourth of L or more (“5”); a method for designing a DNA code, wherein the subword constraint of length m is half of L or more (“6”); a method for designing a DNA code, wherein the set S1 of oligonucleotide sequences is a set of oligonucleotide sequences that contains or never contains a particular subsequence (“7”); a method for designing a DNA code, wherein the codewords of the predetermined error-correcting code are selected from Hamming codes, BCH codes, maximum-length codes, Golay codes, Reed-Muller codes, Reed-Solomon codes, Hadamard codes, Preparata codes, reversible codes, constant-weight codes, or nonlinear codes (“8”); and a method for designing a DNA code, wherein a set of base sequences corresponding to a symbolic unit has a sequence unlike that of natural DNA, and has a constant alignment of [GC][AT] or [CT][AG].

Further, the present invention provides a DNA code consisting of a set of base sequences corresponding to a symbolic unit, which can write optional information into an optional noncoding region not including any DNA genetic information by using a code system decoded by computer (“10”); a DNA code having a constant alignment of [GC][AT] or [CT][AG], and consisting of a set of base sequences designed so that their melting temperatures are standardized in the same predetermined range (“11”); a DNA code consisting of a set of base sequences in which an error such as skip or substitution of some bases is easily detected (“12”); a DNA code comprising an error-correcting function which can decrypt (decode) with high reliability even in the presence of an error such as shift of a reading frame of a base sequence corresponding to a symbolic unit or substitution of plural bases (“13”); a DNA code which does not form a stable secondary structure with base sequences corresponding to a symbolic unit, wherein physical inhibition to inhibit amplification by a primer does not occur in any ligation of letters (“14”); a DNA code consisting of a set of base sequences corresponding to a symbolic unit, which is easily distinguished from natural DNA (“15”); a DNA code, wherein a base alignment is limited in a base sequence, with which whether a specific subsequence appears or not is easily examined (“16”); a DNA code consisting of 112 codewords of length 12, showing mismatches at least at four positions in any hybridization, having at most six consecutive subsequences, and maintaining the same melting temperature in the approximation using the nearest neighbor method (“17”); a DNA code which can be obtained according to any one of the methods for designing described in above (“18”); and a method for writing optional information into DNA, wherein the DNA code is embedded into an optional noncoding region not including any DNA genetic information (“19”).

The present invention still further provides: a method for writing optional information into DNA, wherein the DNA is a vector DNA (“20”); a method for writing optional information into DNA, wherein the DNA is a genomic DNA (“21”); a method for writing optional information into DNA, wherein a DNA creator can be identified by the DNA code (“22”); a labeled vector wherein the DNA codes are embedded into an optional noncoding region not including any DNA genetic information (“23”); a labeled cell, wherein the DNA codes are embedded into an optional noncoding region not including any DNA genetic information (“24”); and a DNA tag having the DNA codes (“25”).

### Effect of the Invention

According to the present invention, DNA codes having following features can be designed.
1. All the letters have the same alignments of GC/AT. This condition allows the DNA codes to share the same melting temperatures and allows the DNA codes to be distinguished from natural DNA easily. Errors such as skip of some bases can be detected easily, too. Further, since all of the letter arrays have the same pattern, a specific base sequence appears in the extremely limited position, so it can be easily detected whether a specific subsequence appears or not.
2. All of the letters are different from each other by bases equal to approximately one-third of length of DNA sequences denoting the letters, and they are also different from each other by bases equal to approximately one-third of concatenation of optional letters including the complementary sequence. This is referred to as an “error-correcting function”, which provides a function to decipher the information strings with high reliability even in the presence of errors such as shift of a reading frame of letter arrays or substitution of plural bases.
3. All of the letters and the ligated part of the letters do not have consecutive match of base sequences of particular length or longer. This condition indicates that the letters do not construct a secondary structure with high stability, and physical inhibition to inhibit amplification by the primer is not induced in any ligation of letter arrays.

### BRIEF DESCRIPTION OF DRAWINGS

FIG. 1 is a view showing that when GC template t of the present invention, which is 110100, is used, then the Hamming distance minimum value MD (t) equals 2, regardless of the way the GC template t is shifted to ligated sequences.

### BEST MODE OF CARRYING OUT THE INVENTION

The method for designing a DNA code of the present invention is not particularly limited to as long as it is a method for constructing a set S1 of oligonucleotide sequences corresponding to a signal unit in signaling, comprising the following steps: 1) selecting a binary string (GC templates) such that its Hamming distance against its reverse sequence, its block shift, and the distance against the overlap part of its tandem concatenation, its concatenation with its reverse sequence, and the tandem concatenation of its reverse sequence are equal to or above the predetermined value k, and in the following, an oligonucleotide sequence of predetermined length n (n is an integer 6 or more) is specified by the binary string of 0 and 1 (GC template) of predetermined length L (L is an integer 6 or more), meaning that the position of G or C ([GC]), or A or T ([AT]) are fixed; 2) selecting a set having a subword constraint of length m as a template from the set of the selected GC templates; and 3) combining codewords of the predetermined error-correcting codes having a subword constraint of length m likewise; or comprising the following steps: 1) selecting a binary string (AG template) such that its Hamming distance against its reverse inverted sequence, its block shift, and the distance against the overlap part of its tandem concatenation, its concatenation with its reverse inverted sequence, and the tandem concatenation of its reverse sequence are equal to or above the predetermined value k, and in the following, an oligonucleotide sequence of length n (n is an integer 6 or more) is specified by the binary string of 0 and 1 (AG template) of predetermined length L (L is an integer 6 or more), meaning that the position of A or G ([AG]), or T or C ([CT]) are fixed; 2) selecting a set having a subword constraint of length m as a template from the set of the selected AG templates; and 3) combining the codewords of predetermined error-correcting codes having a subword constraint of length m likewise. DNA sequence and RNA sequence are included in the above oligonucleotide sequences; “a method for designing an RNA code as an information carrier” is also included in the above “a method for designing a DNA code as an information carrier” for the sake of convenience. Meanwhile, in the present invention, encoding means relating a specific base sequence to letters or symbols in order to process the letters or symbols by computer, while a DNA code is referred to as a set of signal units (letters such as alphabet, which may be called DNA codewords) represented using DNA as a medium. The DNA code which can be obtained by the method for designing of the present invention can be advantageously used when optional information is written into an optional noncoding region such as intron, 5′-noncoding region, or 3′-noncoding region, not including any DNA genetic information.

Upper limit of the predetermined length n (n is an integer 6 or more) of the above oligonucleotide sequences is not limited, but it comprises generally 100 bases, preferably 32 bases, and the subset of the set S1 is also included in the set S1 of the above oligonucleotide sequences for the sake of convenience. Hereinafter, it is described how the DNA codes consisting of a set of base sequences corresponding to a signal unit such as alphabet using the set S1 inducing mismatches is designed with the use of a GC template mainly, focusing the case where the oligonucleotide sequence is a DNA sequence, including the case of complementary sequences.

The P sequences in the above set S1 designed by using a template not only induce mismatches of predetermined value or more between the sequences themselves, and between the P sequences and other P sequences in the set S1, in both cases where sequences are shifted (sequences are staggered) and not shifted and can avoid mishybridization, but also induce mismatches of predetermined value or more between the P sequences and P^{C} sequences which are complementary sequences of each of other oligonucleotide sequences (excluding the P sequences themselves) in the set S1, that is, P^{C} sequences constructed by substituting T, A, C and G for A, T, G and C in the P sequences respectively, and reversing the direction of 5′ and 3′, in both cases where sequences are shifted and not shifted, and can avoid mishybridization. The P sequences further induce mismatches of predetermined value or more between the P sequences and oligonucleotide sequences constructed by ligating each of oligonucleotide sequences in the set S1, that is, ligated sequences of P sequences, and ligated sequences of PC sequences, ligated sequences of P sequences and PC sequences, ligated sequences of PC sequences and P sequences, etc., and can avoid mishybridization. Here, mismatch means a pairing with bases other than complementary bases in hybridization, and as mismatches of predetermined value or more, there is no particular limitation as long as it is the number of mismatches with which mishybridization can be avoided, however, it is preferable that mismatches are one-fifth or more, more preferably one-fourth or more, and most preferably one-third or more of predetermined length n (n is an integer 6 or more) of oligonucleotide sequences.

Further, it is preferable that the oligonucleotide sequence consisting of the above set S1 can be processed as a set of sequences with which it is possible to easily locate the position where a particular subsequence appears. Examples of the particular subsequences include restriction sites; expression signal sequences including poly A portions of RNA, ATG which is a translation initiation codon, TAA, TAG, TGA, etc. which are stop codons; consensus sequences GCCAATCT, ATGCAAAT, recognized by transcription factors, and optional DNA sequence signal such as base sequences encoding variable regions of antibodies.

The afore-mentioned set S1 of oligonucleotide sequences can be usually designed in two steps. A GC template is designed with the use of the Hamming distance at the first step, and the set S1 of oligonucleotide sequences of the present invention as an object is designed using the set of oligonucleotide sequences represented by the designed GC templates by using the theory of error-correcting codes at the next step. It is determined at the first step whether each of the positions in the sequences is [GC] or [AT]. This position is represented by a GC template comprising 0 and 1; b₁ b₂ . . . b₁ (b₁ ε{0, 1}), and 1 and 0 mean [AT] and [GC], respectively, or, 1 and 0 mean [GC] and [AT], respectively. Therefore, not 4^{L} kinds but 2^{L} kinds of sequences are represented by a GC template of length L. At the next step, base sequences are determined by specifically substituting bases [AT] for the position 1, and bases [GC] for the position 0, or bases [GC] for the position 1, and bases [AT] for the position 0 by a GC template.

The Hamming distance mentioned above is used as a scale for similarity between sequences. For example, the Hamming distance between two strings x=x₁, x₂, . . . xₙ and y=y₁, y₂, . . . yₙ is defined as the number of index i that complies with the condition of xᵢ≠yᵢ. In addition, as mishybridization between DNA sequences can occurr even when sequences are shifted (staggered), it is necessary to consider the Hamming distance in the case where sequences are shifted. Since “shift” occurs when one sequence is longer than the other, in case of |x|<|y|, the Hamming distance between the two strings is made to be the minimum value of the Hamming distance between x and each of |y|−|x|+1) subsequences of length |x| contained in y. The Hamming distance indicated by this minimum value can be represented by H (x, y).

Next, function MD (abbreviation of minimum distance) against a GC template t is considered in order to obtain the Hamming distance between a GC template t and ligated sequences of the GC templates t, ligated sequences of reverse sequences t^{R} of the GC templates t, ligated sequences of the GC templates t and reverse sequences t^{R}. The above-mentioned reverse sequence t^{R} of GC template means a sequence wherein a binary string of the GC template t is aligned reversely. As the Hamming distance between a GC template t and a GC template t, its reverse sequence t^{R}, which are sequences at both outer sides of ligated sequences, is already obtained, it is suffice to consider sequences wherein one letter each is deleted from both ends of ligated sequences when obtaining minimum value of the Hamming distance by shifting GC templates t against ligated sequences, consequently, it is convenient to use a symbol [ ] in a mathematical formula of MD (t). The meaning of symbol [ ] is: [s₁ s₂ s₃ . . . sₘ₋₁ sₘ]=s₂ . . . sₘ₋₁, that is, it means a sequence wherein one letter each is deleted from both ends. Therefore, the minimum distance MD (t) of the Hamming distance between GC templates t and ligated sequences is represented by the following formula.
MD(t)=min{H(t, t^{R}), H(t, [tt]), H(t, [tt^{R}]), H(t, [t^{R}t]), H(t, [t^{R}t^{R}])}.

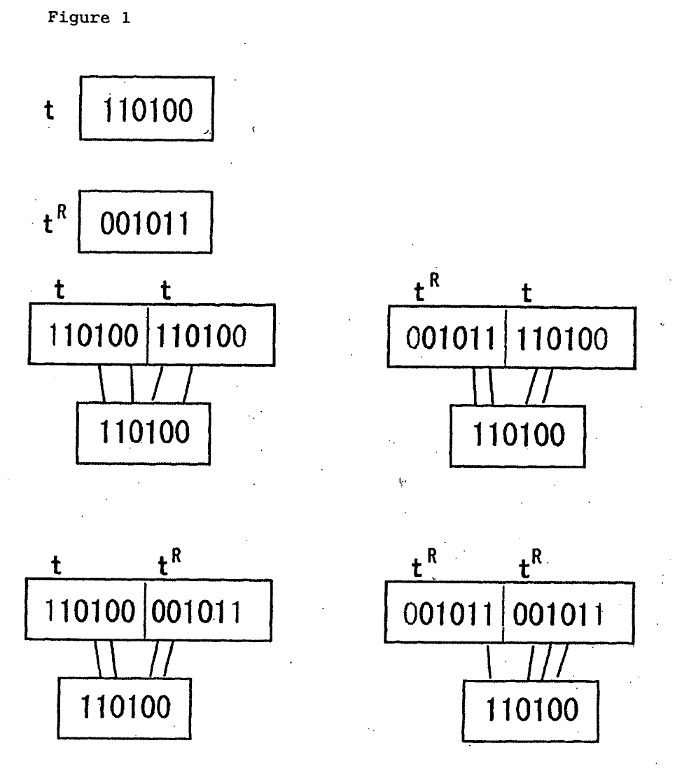

Consequently, in case where MD(t)=k(k≧0) for a GC template t, at least Hamming distance k is ensured for sequences [tt], [tt^{R}], [t^{R}t], [t^{R}t^{R}], including ligating parts thereof, wherein one letter each is deleted from both ends of ligated sequences, when a GC template t is shifted against ligated sequences. FIG. 1 shows that when GC template t=110100, then MD(t)=2. In this case, reverse sequence t^{R}=001011, [tt]=1010011010, [tt^{R}]=1010000101, [t^{R}t]=0101111010, [t^{R}t^{R}]=0101100101, and FIG. 1 shows the case where each Hamming distance is 2. As seen from FIG. 1, GC template t=110100 cannot shorten the Hamming distance beyond 2 regardless of the way of shifting, therefore, it would be defined that MD(t)=2.

Thus, the method for designing a GC template mentioned above is used at the first step of constructing the set S1 of oligonucleotide sequences mentioned above. As seen from the above explanation, the method for designing a GC template is not particularly limited as long as it is a method comprising selection of GC templates such that its Hamming distance against its reverse sequence, its block shift, and the distance against the overlap part of its tandem concatenation, its concatenation with its reverse sequence, and the tandem concatenation of its reverse sequence, are equal to or above the predetermined value k, in the following, an oligonucleotide sequence of predetermined length n is specified by the binary string of 0 and 1 (GC template), meaning that the positions of [GC], or [AT] are fixed. However, the length L of GC template is 6 or more, preferably 6 to 100, more preferably 6 to 32, most preferably around 20, which is often used in experiments of molecular biology. If the length is 5 or less, the one having desired Hamming distance cannot be obtained. By using the GC template having the length L, a set S1 of oligonucleotide sequences of corresponding length n can be obtained. Further, the predetermined value k is not particularly limited as long as it is a value that allows oligonucleotide sequences constructed from the GC template to be the oligonucleotide sequences of the present invention that can avoid mishybridization. The value is preferably one-fifth or more, more preferably one-fourth or more, most preferably one-third or more of the length L of the GC template.

In general, when the length L is increased or MD value (k value) is decreased, many more GC templates will exist, however, a GC template of predetermined length and having the greatest k value (MD value) is particularly important. Examples of GC templates of length L=6 to 32 and having the greatest k value (MD value) include: GC templates having length L=6 to 10, 11 to 15, 16 to 18, 19, 20 to 22 and 24, 23 and 25, 26 and 27, 28 and 29, 30 to 32, and the predetermined value k=2, 4, 6, 7, 8, 9, 10, 11, 12, respectively. The maximum value of the predetermined value k in the GC templates of length L=6 to 32, the number of GC templates having the maximum value, and specific examples are shown in [Table 1]. In addition, the shortest GC templates that fulfill specific MD value (k value) are shown in [Table 2]. Further, specific examples for GC templates of length L=11 to 27 and those for GC templates of length L=28 to 30 are shown in [Table 3] and [Table 4], respectively. In [Table 2], GC templates are enumerated excluding the ones that have the same reverse sequences or sequences wherein 0 and 1 are reversed, and in [Table 3] and [Table 4], “items” are the numbers after omitting GC templates that become identical by cyclic shift.

TABLE 1

| | | The | |
|---|---|---|---|
| | | number | |
| Length | Distance | of | |
| L | k | templates | Specific examples |
| 6 | 2 | 1 | 110100 |
| 7 | 2 | 6 | 0101100 |
| 8 | 2 | 18 | 11001000 |
| 9 | 2 | 45 | 111010000 |
| 10 | 2 | 148 | 0111101000 |
| 11 | 4 | 3 | 11000100101 |
| 12 | 4 | 31 | 111000100101 |
| 13 | 4 | 109 | 0101100010000 |
| 14 | 4 | 496 | 10011010100000 |
| 15 | 4 | 1426 | 111000001010011 |
| 16 | 6 | 12 | 1110001101000100 |
| 17 | 6 | 67 | 00101110010110000 |
| 18 | 6 | 1043 | 111001000110000111 |
| 19 | 7 | 5 | 1111001001100001010 |
| 20 | 8 | 6 | 11101110001000110100 |
| 21 | 8 | 19 | 111101010000001101100 |
| 22 | 8 | 982 | 1110001011101101000000 |
| 23 | 9 | 3 | 11100000101001100110101 |
| 24 | 8 | 71007 | 111101001011000000001111 |
| 25 | 9 | 88 | 1010111100000001010011001 |
| 26 | 10 | 731 | 10101110110110001111000000 |
| 27 | 10 | 4980 | 111010011010100100000010111 |
| 28 | 11 | 18 | 1111101011000000111001010001 |
| 29 | 11 | 1 | 11101110110100100010001110000 |
| 30 | 12 | 178 | 001011111000101101011001100000 |
| 31 | 12 | 2615 | 1001110110111100010101110000000 |
| 32 | 12 | 191945 | 11010011110101000110111000000000 |

TABLE 2

| MD | | | | |
|---|---|---|---|---|
| value | Length | Templates | | |
| 2 | 6 | 110100 | | |
| 4 | 11 | 01000111010, | 00111011010, | 01110100100 |
| 6 | 16 | 1011001000010101 | 1011100000100101 | 1011100010000101 |
| | | 1001111000001001 | 0101101110000010 | 0111101000001100 |
| | | 1110001101000100 | 0011010011101000 | 1011000111001000 |
| | | 0101101110001000 | 0101111000110000 | 1100101101010000 |

| | | |
|---|---|---|
| 7 | 19 | 0111101010000110110, 1001100001010111100, |
| | | 1010111100110110000, 1010111100100110000, |
| | | 1101100111101010000 |
| 8 | 20 | 11010011101110001000, 01111010011001101000, |
| | | 11011101000100111000, 11100011011101000100, |
| | | 11101110001000110100, 11101001100110100001 |

TABLE 3

| Length (d) | |
|---|---|
| 11 (4) | 01110100100 |
| 12 (4) | 000111011010 |
| | 001011100110 |
| | 001111010100 |
| | 010011011100 |
| | 010111100010 |
| | 011010100110 |
| | 101001100000 |
| | 101100001000 |
| | 111001011000 |
| 13 (4) | 0000101100010 |
| 22 items | 0000111011010 |
| | 0001011001110 |
| | 0001011100110 |
| | 0001110110010 |
| | 0010010011100 |
| | 0010100101110 |
| | 0010100111010 |
| | 0010110010110 |
| | 0011110101000 |
| | 0100010111000 |
| | 0110010100000 |
| | 0110011110000 |
| | 0110101001100 |
| | 1000110110100 |
| | 1000111010000 |
| | 1001011100000 |
| | 1010010011000 |
| | 1010110010000 |
| | 1010110110000 |
| | 1010111001000 |
| | 1101100101000 |
| 14 (4) | 79 items |
| 15 (4) | 180 items |
| 16 (6) | 0001100011110100 |
| | 0010011100011010 |
| | 0011010011101000 |
| | 0101000010011011 |
| | 0101101110001000 |
| | 1000001110110100 |
| | 1001111000001001 |
| | 1100101101010000 |
| 17 (6) | 00001000100110111 |
| 26 items | 00001011100101100 |
| | 00010010101100110 |
| | 00010101011011000 |
| | 00011000111110100 |
| | 00011101101001000 |
| | 00100101011111000 |
| | 00100111000101100 |
| | 01000011110110010 |
| | 01000110011110000 |
| | 01001011000101110 |
| | 01001011101100010 |
| | 01001111010101000 |
| | 01010000010011011 |
| | 01100011110100000 |
| | 01110001001101010 |
| | 01110101100101000 |
| | 10000011101010010 |
| | 10011000010111100 |
| | 10110001110010000 |
| | 10110010111000100 |
| | 10111001100010100 |
| | 11000111011010000 |
| | 11010100110100000 |
| | 11101010001100100 |
| | 11110010001100001 |
| 18 (6) | 209 items |
| 19 (7) | 1010111100100110000 |
| 20 (8) | 10000101100110010111 |
| | 11010011101110001000 |
| | 11011101000100111000 |
| 21 (8) | 000101101001111001100 |
| | 001001011011100010110 |
| | 010101000001110011011 |
| | 010101111000110110000 |
| | 011010001010011101100 |
| | 011110100000100110110 |
| | 100110110101110000010 |
| | 101000001100010011110 |
| | 101011110011011000000 |
| | 111100110000011010100 |
| 22 (8) | 409 items |
| 23 (9) | 01111010110011001010000 |
| 24 (8) | 10760 items |
| 25 (9) | 0000100011011010011101010 |
| 20 items | 0000101011000110110100110 |
| | 0000110010101100011110010 |
| | 0001000101101001011100110 |
| | 0001100111100101011010000 |
| | 0010000110110001111010100 |
| | 0010011100001101101010100 |
| | 0010100110001101011110000 |
| | 0011110101100110010100000 |
| | 0101000001100110001111010 |
| | 0101001101001110110001000 |
| | 0101110011010010100110000 |
| | 0110011100010100001011010 |
| | 0110100011000110100000111 |
| | 0111100110010000110101000 |
| | 1000001010001100111010110 |
| | 1011001110010101011000000 |
| | 1101010011100110100010000 |
| | 1110010100110011010100000 |
| | 1110011001000001010110100 |
| 26 (10) | 330 items |
| 27 (10) | 2272 items |

TABLE 4

| | |
|---|---|
| 28 (11) | 0100001111010001111011101000 |
| | 0100011100100100100011111011 |
| | 0111010110001111110010100000 |
| | 0111111001001101001100001010 |
| | 1010101000110000101101001111 |
| | 1011101010010111101000001100 |
| | 1100110010000011101010110011 |
| 29 (11) | 11101110110100100010001110000 |
| 30 (12) | 000000110100101010111100110011 |
| 157 items | 000001000111010111101000011011 |
| | 000001011001011110100011001110 |
| | 000001011111100010110011001010 |
| | 000001110101101010001110110010 |
| | 000010000011011001110010101111 |
| | 000010110101010011111100110000 |
| | 000011001001010110011111110000 |
| | 000011001110000001010101101111 |
| | 000011010010011000111011101100 |
| | 000011011111000110101001110000 |
| | 000011111011001011010100110000 |
| | 000100000110111110011100100011 |
| | 000100001101000011011011101011 |
| | 000100100111000000011010111111 |
| | 000100100111110011100010101100 |
| | 000101000110100111101000111010 |
| | 000101001000100110111110000111 |
| | 000101001011001010111111001000 |
| | 000101001111101000110011101000 |
| | 000101110111100010111100001000 |
| | 000110001001110111100101100100 |
| | 000110100110011000010110101110 |
| | 000110101010100111100110011000 |
| | 000110110100100111111010101000 |
| | 000110111101010100100101110000 |
| | 000111010100001000001101101111 |
| | 000111010101001111101001001000 |
| | 000111111000000100011001011011 |
| | 000111111010101100011010010000 |
| | 001000001010111010111100010011 |
| | 001000010111110011011000011010 |
| | 011001001010100010111110011000 |
| | 011001010111111000000010100110 |
| | 011001011100101011001110010000 |
| | 011001100000011111010110001010 |
| | 011001111100000110001010011010 |
| | 011010000001010111100011011010 |
| | 011010011000001101110011010100 |
| | 011011101000101101001110000100 |
| | 011011101010011000111100000010 |
| | 011101000110010000010011111010 |
| | 011110000100010110100001101110 |
| | 011110000110010001100101010110 |
| | 011110010011001010110110000100 |
| | 011111100010011010011000010100 |
| | 011111101010000001100100101100 |
| | 100000001111010101100011100110 |
| | 100000011110010110111001100100 |
| | 100001000010011010001011110111 |
| | 100001010110010000011100111110 |
| | 100001101001111011000101001100 |
| | 100010000110111110011101000100 |
| | 100010011100000100010111010111 |
| | 100010100111011011010010010001 |
| | 100100000011110101100011101100 |
| | 100100001010110111000111100100 |
| | 100101011110110010111000100000 |
| | 100101101111000111010001100000 |
| | 100101111011100010000101001001 |
| | 100110000001010111100010111100 |
| | 100110000001101001010101100111 |
| | 100110001101011111001001000001 |
| | 101000001001101111100011010100 |
| | 101000101011010111110000010001 |
| | 101001001101111100011000000101 |
| | 101001100011111101010100000001 |
| | 101001101001111110000001010001 |
| | 101001110010000110000101010111 |
| | 101010100111011011010000010001 |
| | 101100001000100111011010001110 |
| | 101100101010000100011001111100 |
| | 101100111011011100000011000100 |
| | 001000011101000011011011110100 |
| | 001000100110111011110000010110 |
| | 001000110010111110000101010110 |
| | 001001000110001111011011101000 |
| | 001001001111000010111011100010 |
| | 001001100000111001101111010100 |
| | 001010001100101011110111010000 |
| | 001010100110000110100111111000 |
| | 001010101110011110100101100000 |
| | 001010101111110010010100110000 |
| | 001010111101001101010011010000 |
| | 001011100100000101001111011100 |
| | 001011100110010111110001010000 |
| | 001011110111010011000101001000 |
| | 001011111000101101011001100000 |
| | 001100100011101101001000111100 |
| | 001100110000111101010001001011 |
| | 001100110110100100010101111000 |
| | 001101110001000100101100111100 |
| | 001110000100100101011011111000 |
| | 001110101000010010010011110110 |
| | 001110110111010100010010001100 |
| | 001111001000110101101100100100 |
| | 001111010000100010001011101110 |
| | 001111100110001010101101001000 |
| | 001111110101000010001100101100 |
| | 010000101010111011011000001110 |
| | 010000110111010001101010011100 |
| | 010001000010111000101110011011 |
| | 010001000111011101101000011010 |
| | 010001011001100010000111101011 |
| | 010001100111010011011010101000 |
| | 010001101011000011011101000110 |
| | 010010000110000111010001111011 |
| | 010010100111011111000001100010 |
| | 010010110101000111110011001000 |
| | 010011011111100010100111000000 |
| | 010100111101011100000011001100 |
| | 010101100010000110100110101110 |
| | 010111100001100001010111011000 |
| | 010111100011000010010011011100 |
| | 010111100100110000010101111000 |
| | 101100111111000000110100101000 |
| | 101101101110001010011101000000 |
| | 101101110011000100010010111000 |
| | 101110000101111001101000100001 |
| | 101110001101010011110000001001 |
| | 101110100011110011100100001000 |
| | 101111001100001011001010101100 |
| | 101111010001000010011010001101 |
| | 101111010001001001101000110001 |
| | 101111010001101011100010010000 |
| | 101111010101010001100000100101 |
| | 101111100011001100110000010010 |
| | 110000001000110001101101001111 |
| | 110000001001001100011100101111 |
| | 110001110101001101010000100011 |
| | 110010000000110001010111001111 |
| | 110010000100101000111101101100 |
| | 110010100100000111000111101100 |
| | 110010111101000010010001010011 |
| | 110100111011010001110110000000 |
| | 110100111011101000100011000010 |
| | 110101001100111101000000110010 |
| | 110101100100001001110000101011 |
| | 110110011001000000101011110001 |
| | 110110110001010111100000011000 |
| | 110111010011000001000110111000 |
| | 110111100001000110100001110100 |
| | 111000001101110110101100001000 |
| | 111000010111101110100010000100 |
| | 111000111001101101010010000010 |
| | 111001000001111001101011000010 |
| | 111001000011001100000111001011 |
| | 111001001011011100110001000001 |
| | 111001010111000110001000000111 |
| | 111001011000100101010001000111 |
| | 111001111100000010001010011010 |
| | 111011000001001010001010100111 |
| | 111011010001001010011010000110 |
| | 111100101101000000101110011000 |
| | 111100101110011000000101000101 |
| | 111110010101100001011010001000 |
| | 111110011001010001100000011010 |

The GC template sequences enumerated in [Table 1] to [Table 4], etc., can be selected by searching exhaustively all patterns from sequences comprising only 0 to sequences comprising only 1, by a person skilled in the art. However, there is no need to search all 2^{L} patterns to find a GC template of length L. It is suffice to take into account the GC templates wherein bit 1 contained therein is L/2 or less because GC templates whose bits 01 are reversed have same property. In addition, from the constraint of the number of mismatches, it is shown that in case where the minimum distance is d, the number of bit 1 is at least (L−sqrt(L²−2 dL))/2 (sqrt means square root). The GC templates can be efficiently obtained by using these constraints additionally. Further, when GC templates are designed such that the set S1 of oligonucleotide sequences constructed from GC templates is made to be a set of oligonucleotide sequences that contains or never contains particular subsequences such as restriction sites mentioned above, such designing corresponds to the narrowing of the space for exhaustive search, and therefore it contributes to easier designing.

Following to the step of designing GC templates by using the Hamming distance mentioned above, the set S1 of oligonucleotide sequences mentioned above can be designed at the step in which the theory of error-correcting codes are used from the set of oligonucleotide sequence represented by the designed GC templates, that is, by combining codewords of any error-correcting code. As for the codewords of error-correcting codes mentioned above, any codewords can be used as long as they are known codewords of error-correcting codes, and specific examples include Hamming codes, BCH codes, maximum-length codes, Golay codes, Reed-Muller codes, Reed-Solomon codes, Hadamard codes, Preparata codes, reversible codes, constant-weight codes, and nonlinear codes.

The motive for using the theory of error-correcting codes is to ensure mismatches to complementary sequences in case where there is no shift. Therefore, as to the set S1 in consideration of reverse sequence, it is not always necessary to use error-correcting codes. Error-correcting codes are a set of codewords wherein there are at least a certain number of mismatches between optional codewords. In case of preventing mishybridization between a set S1 and a set of reverse sequences thereof, it is only necessary to apply a set of codewords wherein there are at least a certain number of matches (not mismatches) between optional codewords. As for the set S1 of oligonucleotide sequences mentioned above, information of the codewords and GC templates are reflected on the sequences. Therefore, it is suffice to use error-correcting codes maintaining the Hamming distance (the number of mismatches) k or more in order to ensure k mismatches to complementary sequences, and it is suffice to use codes maintaining the number of matches k or more in order to ensure k mismatches to reverse sequences.

In the theory of error-correcting codes, codes wherein a redundant bit for detecting and correcting errors, which is called parity bit, is added to a given information bit to make the Hamming distance between optional codewords a certain value or above, have been developed. The minimum value of the Hamming distance between codewords is called minimum distance. As the object of the code theory is to design the one that maintains the minimum distance largely and contains many codewords, there are many codes that meet the purpose of the present invention. For example, there are 4096 words of Golay codes of code length 23 and minimum distance 7. With the use of this code, it is possible to design 4096 oligonucloetides for one GC template of length 23 (MD value is up to 9).

In order to prepare oligonucleotide sequence fulfilling stricter constraints, for general-purpose DNA codes, a subword constraint of length m should be considered together when a template used in set S1 mentioned above is selected. When the set is selected, binary string of 0 and 1 is designed so that it is presenct consecutively m or more between templates constructing a set S1, and the distance between codewords is designed so that the binary string does not match consecutively m or more between codewords by using obvious transformation to the Max Clique Problem from error-correcting codewords. As for m value in subword constraint of length m, the value 10 or less is preferable in that mismatches can be fully dispersed. When L is 12, 7 can be exemplified as m value.

For instance, combining 001110010000, 001001010100, 000000000000, 010001110101, 111010011000 (lower) as the codewords of nonlinear codes of length L=12 having a subword constraint of minimum distance 4, length 7 with 000110011101 and 001010111100 (upper) of length L=12 having a subword constraint of MD(t)=4, length 7 as for a template in a set S1, results that the obtained bases induce at least four mismatches against any concatenations, sifts, in which 7 bases or more of base sequences not inducing mismatches is not present consecutively. For instance, when 00 is A, 01 is T, 10 is G, and 11 is C, ten sets of DNA sequences consisting of 12 bases shown in Table 5 whose GC content is ½ are obtained. Further, when 00 is G, 01 is C, 10 is A, and 11 is T, ten sets of DNA sequences consisting of 12 bases shown in Table 6 whose GC content is ½ are obtained.

TABLE 5

000110011101 000110011101 0001100
001110010000 001001010100 0000000
AATCCAACGTAG AATGGTACGCAG AAAGGAA
11101 000110011101 000110011101
00000 010001110101 111010011000
GGGAG ATAGCTTCGCAC TTTGCAACCGAG
001010111100 001010111100 0010101
001110010000 001001010100 0000000
AACTCAGCGTAA AACAGTGCGCAA AAGAGAG
11100 001010111100 001010111100
00000 010001110101 111010011000
GGGAA ATGAGTCCGCAT TTCACAGCCGAA

TABLE 6

000110011101 000110011101 0001100
001110010000 001001010100 0000000
GGCTTGGTAAGA GGCAACGTATGA GGGAAGG
11101 000110011101 000110011101
00000 010001110101 111010011000
AAAGA GCGAACCTATGT CCCATGGTTAGA
001010111100 001010111100 0010101
001110010000 001001010100 0000000
GGTCTGATAAGG GGTGACATATGG GGAGAGA
11100 001010111100 001010111100
00000 010001110101 111010011000
AAAGG GCAGACTTATCC CCTGTGATTAGG

Next, the DNA code of the present invention is not particularly limited as long as it can write optional information into an optional noncoding region not including any DNA genetic information by using a code system decodable by computer such as binary code and the DNA code consists of a set of encoded base sequences, but followings are preferable: a DNA code consisting of a set of base sequences which is encoded so that not only GC content but also alignment of GC bases are same and the melting temperatures estimated by approximation using the nearest neighbor method used in experiments of molecular biology are in the predetermined range, a DNA code consisting of a set of encoded base sequences in which an error such as skip or substitution of some bases is easily detected, a DNA code comprising an error-correcting function which can decode with high reliability even in the presence of an error such as an shift of reading frame of encoded base sequences or substitution of plural bases, a DNA code which does not form a stable secondary structure with encoded base sequences, wherein physical inhibition to inhibit amplification by a primer does not occur in any ligation of codewords, a DNA code consisting of a set of encoded base sequences corresponding to letters, which can be easily distinguished from natural DNA, and a DNA code wherein a base alignment is limited and appearance of a specific subsequence can be easily located. The DNA code can be obtained by the method for designing DNA code of the present invention. A DNA code consisting of 112 codewords of length 12, which induces mismatches at least at four positions between codewords in any ligation of codewords including their complementary sequences and at most 6 consecutive matches of bases prevents mishybridization, and further maintains the same melting temperature in approximation using the nearest neighbor method, can be cited as a specific example.

As for method for writing optional information by using the DNA of the present invention, it is not specifically limited as long as it is a method wherein the DNA code of the present invention mentioned above, consisting of a set of base sequences corresponding to letters such as alphabet, is embedded into an optional noncoding region such as intron, 5′-noncoding region, or 3′-noncoding region, not including any DNA genetic information. As for the DNA in which the DNA code of the present invention is embedded, a vector DNA such as a plasmid vector DNA and a viral vector DNA, and a genomic DNA of animal or plant cell and microbial cell can be exemplified. The method for writing optional information into the DNA of the present invention allows DNA signature by embedding DNA codes corresponding to letters such as alphabet with which the creator can be identified, into an optional noncoding region not including any DNA genetic information. The present invention also relates to a labeled vector or labeled cells in which the DNA code of the present invention is embedded in an optional noncoding region not including any DNA genetic information, and with which the creator can be identified.

Though plural types of oligonucleotide strands consisting of the DNA codes of the present invention are fixed in high density on a substrate, the sequences do not often cause mishybridization each other; consequently, the set of encoded base sequences of the present invention can be advantageously applied in DNA tip or RNA tip, or as DNA tag or RNA tag. Further, they do not often cause mishybridization with their complementary sequences, so the set of encoded base sequences of the present invention are useful as primers in PCR or the like. Moreover, since the set of encoded base sequences of the present invention can be easily proved that they do not have particular subsequences such as restriction site in addition to that they do not often cause mishybridization, it can be advantageously used in DNA computing system comprising following steps: artificially synthesizing DNA sequences in which various symbol manipulation operating systems such as logical expression and graph structure are recorded, and cutting and pasting the sequences according to protocols of molecular biological experiments, in which sequences obtained at the end of the experiments are “calculation results” of DNA computing.

### EXAMPLE

The present invention is described below more specifically with reference to Example, however, the technical scope of the present invention is not limited to the following exemplification.

(DNA ASCII Code)

When the design of the ASCII code (128 letters) using DNA is considered, one DNA codeword is used for each of the letters such as alphabet. One of shorter error-correcting codes with at least 128 codes is the nonlinear (12,144,4) code (Sloane, N. J. A. and MacWilliams, F. J.: The Theory of Error-Correcting Codes. Elsevier, 1997). The above notation (12,144,4) reads ‘a length-12 code of 144 words with the minimum distance 4’ (one error-correcting, two error-detecting). By using a Max Clique Problem solver (http://rtm.science.unitn.it/intertools/) among 144 words, 32, 56, and 104 words can be selected which satisfy the length 6, −7, and −8-subword constraints, respectively. The code represented by (12,144,4) is shown in Table 7, and codewords with dagger among 144 codewords are 56 codewords satisfying the length 7-subword constraint.

TABLE 7

| | | | | | |
|---|---|---|---|---|---|
| 110010100000 | 110001010000^{†} | 110000001010 | 110000000101 | 101100100000^{†} | 101001001000^{†} |
| 101000010001 | 101000000110^{†} | 100101000100^{†} | 100100011000 | 100100000011 | 100011000010 |
| 100010010100 | 100010001001 | 100001100001^{†} | 100000110010 | 100000101100^{†} | 011100000010 |
| 011010000100 | 011000110000^{†} | 011000001001 | 010110001000 | 010100100100 | 010100010001 |
| 010011000001 | 010010010010 | 010001101000 | 010001000110 | 010000100011^{†} | 010000011100 |
| 001110010000^{†} | 001101000001^{†} | 001100001100 | 001010101000^{†} | 001010000011 | 001001100010 |
| 001001010100^{†} | 001000100101 | 001000011010^{†} | 000110100010 | 000110000101 | 000101110000^{†} |
| 000101001010 | 000100101001^{†} | 000100010110 | 000011100100 | 000011011000 | 000010110001^{†} |
| 000010001110 | 000001010011 | 000001001101^{†} | 001101011111 | 001110101111 | 001111110101 |
| 001111111010 | 010011011111^{†} | 010110110111^{†} | 010111101110^{†} | 010111111001 | 011010111011^{†} |
| 011011100111 | 011011111100 | 011100111101^{†} | 011101101011 | 011101110110 | 011110011110^{†} |
| 011111001101 | 011111010011^{†} | 100011111101^{†} | 100101111011 | 100111001111^{†} | 100111110110 |
| 101001110111 | 101011011011 | 101011101110 | 101100111110 | 101101101101 | 101110010111 |
| 101110111001 | 101111011100^{†} | 101111100011 | 110001101111 | 110010111110^{†} | 110011110011^{†} |
| 110101010111 | 110101111100^{†} | 110110011101 | 110110101011^{†} | 110111011010^{†} | 110111100101 |
| 111001011101 | 111001111010 | 111010001111 | 111010110101 | 111011010110^{†} | 111011101001 |
| 111100011011 | 111100100111 | 111101001110^{†} | 111101110001 | 111110101100 | 111110110010^{†} |
| 000000000000^{†} | 111111111111^{†} | 000000111111 | 000011101011^{†} | 000101100111 | 000110011011^{†} |
| 000110111100 | 001001111001 | 001010011101 | 001010110110 | 001100110011^{†} | 001111000110^{†} |
| 010001110101^{†} | 010010101101^{†} | 010100001111^{†} | 010100111010 | 010111010100 | 011000010111 |
| 011000101110 | 011011001010^{†} | 011101011000^{†} | 011110100001 | 111111000000 | 111100010100^{†} |
| 111010011000^{†} | 111001100100 | 111001000011^{†} | 110110000110 | 110101100010 | 110101001001 |
| 110011001100 | 110000111001^{†} | 101110001010^{†} | 101101010010^{†} | 101011110000 | 101011000101^{†} |
| 101000101011 | 100111101000 | 100111010001 | 100100110101^{†} | 100010100111^{†} | 100001011110 |

There are 74 GC templates of length 12, the minimum distance 4; 31 templates among them, wherein the reverse sequence and 01 inversion are regarded as the same, are shown in Table 8. Since 128 codewords cannot be derived from a single template under the subword constraint, the pairs of templates are selected. The two pairs of templates induce mismatches in at least four positions in any ligation, and they do not share a subsequence of length 7 or longer. Such eight pairs of templates are shown in Table 9. DNA codewords prepared from these template pairs show even GC base-distribution when they are ligated. Under this condition, DNA codes derived from these templates share close melting temperatures (New Generation Computing 20, 3, 263-277, 2002).

TABLE 8

| | | | | | |
|---|---|---|---|---|---|
| 101001100000 | 011001010000 | 101101110000 | 101100001000 | 011101101000 | 110011101000 |
| 001010011000 | 101110011000 | 111001011000 | 010110111000 | 001101000100 | 011101100100 |
| 001111010100 | 001110110100 | 111010001100 | 110010101100 | 101111000010 | 111001100010 |
| 010111100010 | 111100010010 | 011000001010 | 011010100110 | 100001110110 | 100100011110 |
| 111010010001 | 110110010001 | 100110101001 | 101110000101 | 111000100101 | 110101000011 |
| 110100100011 | | | | | |

TABLE 9

| | |
|---|---|
| 000110011101 and 001010111100 | 000110011101 and 001111010100 |
| 001010111100 and 101110011000 | 001111010100 and 101110011000 |
| 010001100111 and 110000101011 | 010001100111 and 110101000011 |
| 110000101011 and 111001100010 | 110101000011 and 111001100010 |

By combining one of eight template pairs shown in Table 9 with the 56 codewords satisfying the length 7-subword constraint shown in Table 7, 112 codewords (10 of 112 codewords are shown in Tables 5 and 6) were obtained that satisfy the following conditions.
Mismatches are induced at least four positions between any pair of codewords and their complements.
The four mismatches are guaranteed under any shift and concatenation with themselves and their complements (comma-free of index 4).
A subsequence of length 7 or longer is not shared under any shift and concatenation.
All codes have close melting temperatures in approximation using the nearest neighbor method.
Because all codes are derived from only two templates, the occurrence of specific subsequence can be easily located. In addition, the avoidance of specific subsequences is also easy.

The number of codewords thus designed, 112, falls short of the 128 ASCII characters. However, some characters are usually unused in ASCII characters. For example, the values of HTML characters from &#14 to &#31 are not used. Therefore, the 112 codewords suffice for representing DNA ASCII code. This compromise is preferable to loosening of the constraints to obtain 128 codes.

The current status of information-encoding models using DNA was reviewed and the necessity and problems in constructing DNA codes was described. The method for designing a DNA code of the present invention can provide 112 DNA codewords of length 12 and comma-free index 4. The DNA code of the present invention considers optional concatination between codes including their complementary strands, and the DNA code has never been known until today.

1. A method for designing a DNA code, comprising the following steps:
1) selecting a binary string comprising a GC template or an AG template such that its Hamming distance against its reverse sequence, its block shift, and the distance against the overlap part of its tandem concatenation, its concatenation with its reverse sequence, and the tandem concatenation of its reverse sequence are equal to or above the predetermined value k, and in the following, an oligonucleotide sequence of predetermined length n (n is an integer 6 or more) is specified by the binary string of 0 and 1 (GC template or AG template) of predetermined length L, wherein L is an integer of 6 or more, meaning that the position of G or C ([GC]), or A or T ([AT]), or A or G ([AG]), or T or C ([CT]) are fixed; 2) selecting a set having a subword constraint of length m as a template from the set of the selected GC or AG templates; and 3) constructing a set S1 of the oligonucleotide sequences by combining codewords of the predetermined error-correcting codes having a subword constraint of length m likewise.

2. (canceled)

3. The method for designing a DNA code of claim 1, wherein any of oligonucleotide sequences of the set S1, of which Hamming distance is kept equal to or above k, induces mismatches equal to or above the predetermined value against any of the sequences in the set S1, their complementary sequences, sequences constructed by shifting these sequences, and sequences produced by ligation of sequences, of their complementary sequences, and of the sequences and their complementary sequences, and wherein the sequence in the set S1 can avoid mishybridization between them, their complementary sequences, sequences constructed by shifting these sequences, and sequences produced by ligation of the sequences in the set S1, of their complementary sequences, and of the sequences and their complementary sequences, and which facilitates decoding information.

4. The method for designing a DNA code of claim 1, wherein the set S1 of oligonulcleotide sequences of predetermined length n is a set S1 of oligonucleotide sequences of length 32 or less.

5. The method for designing a DNA code of claim 1, wherein the predetermined value k of said Hamming distance is one-fourth of L or more.

6. The method for designing a DNA code of claim 1, wherein the subword constraint of length m is half of L or more.

7. (canceled)

8. The method for designing a DNA code of claim 1, wherein the codewords of the predetermined error-correcting code are selected from Hamming codes, BCH codes, maximum-length codes, Golay codes, Reed-Muller codes, Reed-Solomon codes, Hadamard codes, Preparata codes, reversible codes, constant-weight codes, or nonlinear codes.

9. The method for designing a DNA code of claim 1, wherein a set of base sequences corresponding to a symbolic unit has a sequence unlike that of natural DNA, and has a constant alignment of [GC][AT] or [CT][AG].

10. A DNA code consisting of a set of base sequences corresponding to a symbolic unit, which can write optional information into an optional noncoding region not including any DNA genetic information by using a code system decoded by computer.

11. The DNA code of claim 10, which has a constant alignment of [GC][AT] or [CT][AG], and consists of a set of base sequences designed so that their melting temperatures are standardized in the same predetermined range.

12. The DNA code of claim 10, which consists of a set of base sequences in which an error such as skip or substitution of some bases is easily detected.

13. The DNA code of claim 10, which comprises an error-correcting function decrypting with high reliability even in the presence of an error such as shift of a reading frame of a base sequence corresponding to a symbolic unit or substitution of plural bases.

14. The DNA code of claim 10, which does not form a stable secondary structure with base sequences corresponding to a symbolic unit, wherein physical inhibition to inhibit amplification by a primer does not occur in any ligation of letters.

15. The DNA code of claim 10, which consists of a set of base sequences corresponding to a symbolic unit, and is easily distinguished from natural DNA.

16. The DNA code of claim 10, wherein a base alignment is limited in a base sequence, with which whether a specific subsequence appears or not is easily examined.

17. The DNA code of claim 10, which consists of 112 codewords of length 12, shows mismatches at least at four positions in any hybridization, has at most six consecutive subsequences, and maintains the same melting temperature in the approximation using the nearest neighbor method.

18. A DNA code consisting of a set of base sequences corresponding to a symbolic unit, which can write optional information into an optional noncoding region not including any DNA genetic information by using a code system decoded by computer, said DNA code designed by a method comprising the following steps: 1) selecting a binary string comprising a GC template or an AG template such that its Hamming distance against its reverse sequence, its block shift, and the distance against the overlap part of its tandem concatenation, its concatenation with its reverse sequence, and the tandem concatenation of its reverse sequence are equal to or above the predetermined value k, and in the following, an oligonucleotide sequence of predetermined length n (n is an integer 6 or more) is specified by the binary string of 0 and 1 (GC template or AG template) of predetermined length L, wherein L is an integer of 6 or more, meaning that the position of G or C ([GC]), or A or T ([AT]), or A or G ([AG]), or T or C ([CT]) are fixed; 2) selecting a set having a subword constraint of length m as a template from the set of the selected GC or AG templates; and 3) constructing a set S1 of the oligonucleotide sequences by combining codewords of the predetermined error-correcting codes having a subword constraint of length m likewise.

19. A method for writing optional information into DNA, wherein the DNA code of claim 10 is embedded into an optional noncoding region not including any DNA genetic information.

20. The method for writing optional information into DNA of claim 19, wherein the DNA is a vector DNA.

21. The method for writing optional information into DNA of claim 19, wherein the DNA is a genomic DNA.

22. The method for writing optional information into DNA of claim 19, wherein a DNA creator can be identified by the DNA code.

23. A labeled vector, wherein the DNA code of claim 10 is embedded into an optional noncoding region not including any DNA genetic information.

24. A labeled cell, wherein the DNA code of claim 10 is embedded into an optional noncoding region not including any DNA genetic information.

25. A DNA tag having the DNA code of claim 10.


SEQUENCE LISTING (20 sequences)

SEQ ID NO: 1 - DNA, 12 bp, Artificial
  Oligonucleotide sequence
aatccaacgtag

SEQ ID NO: 2 - DNA, 12 bp, Artificial
  Oligonucleotide sequence
aatggtacgcag

SEQ ID NO: 3 - DNA, 12 bp, Artificial
  Oligonucleotide sequence
aaaggaagggag

SEQ ID NO: 4 - DNA, 12 bp, Artificial
  Oligonucleotide sequence
ataggttcgcac

SEQ ID NO: 5 - DNA, 12 bp, Artificial
  Oligonucleotide sequence
tttgcaaccgag

SEQ ID NO: 6 - DNA, 12 bp, Artificial
  Oligonucleotide sequence
aactcagcgtaa

SEQ ID NO: 7 - DNA, 12 bp, Artificial
  Oligonucleotide sequence
aacagtgcgcaa

SEQ ID NO: 8 - DNA, 12 bp, Artificial
  Oligonucleotide sequence
aagagaggggaa

SEQ ID NO: 9 - DNA, 12 bp, Artificial
  Oligonucleotide sequence
atgagtccgcat

SEQ ID NO: 10 - DNA, 12 bp, Artificial
  Oligonucleotide sequence
ttcacagccgaa

SEQ ID NO: 11 - DNA, 12 bp, Artificial
  Oligonucleotide sequence
ggcttggtaaga

SEQ ID NO: 12 - DNA, 12 bp, Artificial
  Oligonucleotide sequence
ggcaacgtatga

SEQ ID NO: 13 - DNA, 12 bp, Artificial
  Oligonucleotide sequence
gggaaggaaaga

SEQ ID NO: 14 - DNA, 12 bp, Artificial
  Oligonucleotide sequence
gcgaacctatgt

SEQ ID NO: 15 - DNA, 12 bp, Artificial
  Oligonucleotide sequence
cccatggttaga

SEQ ID NO: 16 - DNA, 12 bp, Artificial
  Oligonucleotide sequence
ggtctgataagg

SEQ ID NO: 17 - DNA, 12 bp, Artificial
  Oligonucleotide sequence
ggtgacatatgg

SEQ ID NO: 18 - DNA, 12 bp, Artificial
  Oligonucleotide sequence
ggagagaaaagg

SEQ ID NO: 19 - DNA, 12 bp, Artificial
  Oligonucleotide sequence
gcagacttatgc

SEQ ID NO: 20 - DNA, 12 bp, Artificial
  Oligonucleotide sequence
cctgtgattagg